(19) United States
(12) Patent Application Publication
(10) Pub. No.: US 2004/0047948 A1
(43) Pub. Date: Mar. 11, 2004

(54) Nutritionally enhanced composite food product

(76) Inventors: Thomas A. Arrendale III, Baldwin, GA (US); Chester L. Crum JR., St. Petersburg, FL (US)

Correspondence Address: JINAN GLASGOW, P O BOX 28539, RALEIGH, NC, 276118539

(21) Appl. No.: 10/236,215

(22) Filed: Sep. 6, 2002

(51) Int. Cl. A61K 31/70

(52) U.S. Cl. 426/92

(57) ABSTRACT

A nutritionally-enhanced composite food product for providing a predictable nutrient amount, including at least one serving element, a serving element composed of a first carrier meat and a second nutritionally enhanced foodstuff distinct from the first carrier meat; the first carrier meat having at least one predetermined dimension providing a portion size for providing predictable nutrient levels of the composite food product; thereby permitting nutritional claims to be made to the composite food product for inclusion on packaging and/or labeling.

### BACKGROUND OF THE INVENTION

(1) Field of the Invention

The present invention relates generally to food products and, more particularly, to a nutritionally enhanced composite food product.

(2) Description of the Prior Art

Advancements in the area of nutrition has led to the recognition of the need for supplementing diets with various nutrients, including vitamins, minerals, essential fatty acids, and the like. Many persons consume large amounts of these nutrients in the form of pills, tablets, and liquids. However, taking the large amount of nutrients in this manner is annoying and frequently forgotten, thus returning the person to a state of malnourishment. Therefore, a better way of delivering nutrients to persons is needed. Fortification of foods is another way of delivering nutrients. Fortification of bread with vitamins is even mandated by the US Federal government. Additionally, the USDA carefully restricts food product labeling for meats and has heretofore not permitted nutrition claims on meat products. However, over-consumption of certain nutrients can occur, if the consumer is not aware of the fortification level of foods. Calcium over-consumption has been suggested to occur, especially in men, due to the increased level of calcium in calcium-fortified foods. Meats, which are an excellent source of protein and part of most diets, could be fortified with various nutrients. However, government regulatory agencies, specifically the USDA, currently prohibit the nutritional fortification of meats directly. Such products can not be labeled as fortified, nor the fortifying nutrients described on the label, and as such, the consumers could not make informed decisions regarding such a product within the context of a nutritionally balanced diet. Thus a need exists for a nutritionally-enhanced composite meat product with corresponding nutritional labeling on its packaging so that a consumer can make informed choices regarding the product within the context of a balanced diet.

### SUMMARY OF THE INVENTION

The present invention is directed to a nutritionally enhanced composite food product including a first core or carrier meat with a substantially consistent size and a second nutritionally enhanced foodstuff combined such that the nutritional value of the composite meat product is substantially equal to a target value.

### DETAILED DESCRIPTION OF THE PREFERRED EMBODIMENTS

In the following description, like reference characters designate like or corresponding parts throughout the several views. Also in the following description, it is to be understood that such terms as “forward,” “rearward,” “front,” “back,” “right,” “left,” “upwardly,” “downwardly,” and the like are words of convenience and are not to be construed as limiting terms.

As used within this application, the term “stratified” is used to indicate a separate and distinct component that exists separately without intermixing with another component, which may be layered, e.g., a coating layer is applied onto a carrier layer or component, and the like, which remains separate and distinct without mixing with each other. Also, as used within this application, the term “composite” is used to indicate a structure or product made up of distinct components to form a complex product in which two or more distinct, complementary food substances or components, specifically a meat component and a foodstuff component with at least one nutritional enhancer or additive, are combined without mixing to produce properties in the overall combined product that are not present in either of the two separate components independently.

The composite food product consists of a first core, substrate, or carrier meat, to which a second nutritionally enhanced foodstuff including at least one nutritional enhancer, wherein the foodstuff is stratified from first carrier meat, i.e., the foodstuff and its at least one nutritional enhancer is combined with but exists separately therefrom without mixing therewith.

The carrier meat is preferably selected from any of a variety of meats, including, but not limited to chicken, turkey, beef, lamb, pork, fish, shellfish, and the like. Additionally, the meat may be from animals raised under special conditions to reduce the risk of disease or toxin transmission to humans, such as antibiotic-free, using organic feeds, using all-vegetable diets, and/or without the use of stimulants and hormone. Such meats are stereotypically considered to be preferred by health-conscious consumers as well as animal rights advocates. Furthermore, the animals may also be raised in a low stress environment in ways which reduce stress, and with adequate shelter, comfortable resting areas, sufficient space, proper facilities, and the ability to express normal behavior.

The meat is preferably fully cooked, such that the food product does not require lengthy preparation and there is less risk of microbial spoilage; however, in an alternate embodiment, the composite food product may be partially cooked or uncooked. The nutritionally enhanced composite food product includes a second nutritionally enhanced foodstuff including at least one nutritional enhancer combined, mixed, incorporated, or integrated therewith. The second foodstuff is fortified with at least one nutritional enhancer and is combined with the meat carrier so that the second foodstuff and its at least one nutritional enhancer are maintained separate and distinct from the first meat carrier, i.e., no intermixing occurs between the meat carrier and the added foodstuff with its at least one nutritional enhancer. Nutritional fortification applies only to the second foodstuff. The second foodstuff is distinct from the first carrier meat component, and may be layered therewith, e.g., in the form of an exterior or interior application of the foodstuff onto or into the meat carrier, respectively. The second foodstuff can be on the exterior of the core meat component, such as a coating or breading. Where the foodstuff is located on the exterior surface of the meat component, the foodstuff may be applied onto the meat either before it is cooked or afterward, where the meat is fully or partially cooked prior to packaging and distribution. Alternatively or additionally, the second foodstuff can be in the interior of the first meat component, i.e., the second foodstuff is surrounded by the meat carrier or component. By way of example and not limitation, the second foodstuff may be wrapped with or injected into the carrier.

According to the present invention, nutritional enhancers or additives that are preferably used to fortify the second foodstuff include vitamins and minerals, such as calcium, iron, riboflavin, niacin, folate, thiamin, and the like, essential fatty acids, such as omega-3 and omega-6 fatty acids, amino acids, such as arginine; proteins, such as digestive enzymes, e.g., papain; and carbohydrates, such as glucosamine and chondroitin. In general, molecules that are essential nutrients or function as biomediators to influence the health and growth of the human body may be used as nutritional additives. The nutrients may be in there simplest chemical state, or chemically bound with other molecules. For example, essential fatty acids, such as docosahexaenoic acid, eicosapentaenoic acid, linolenic acid and linoleic acid, may be provided as such or as oils such as fish oil and/or flax seed oil.

The carrier meat has a predetermined dimension size that provides a predictable, controlled element size to enable and ensure that the consumer is receiving a predictable amount of desired nutrients. The composite food product thus provides substantially equal serving sizes and substantially predictable serving sizes, thereby providing predictable nutrient levels and thus permitting nutritional claims to be made to the product for inclusion on packaging and/or labeling.

The RDA values or similar may be the nutritional target level for the composite food product. To achieve this target level, the food product must have the first component of a target size and/or dimensions. The target size and/or dimensions include a 3D shape and/or a surface area or a target weight. By controlling the size and/or shape of the first core component, the amount of the second fortifying component can be added in a predictable, controllable manner for ensuring the predetermined nutritional characteristics of each unit or element, portion size, and/or serving size of the overall composite food product. For example, a nutritionally fortified composite food product consisting of a meat combined with a separate, distinct breading coverage according to the present invention is preferably made such that the amount of breading included in a single unit of the food product is substantially predictable and therefore the nutritional fortification provided by the breading to a single unit is substantially predictable. Thus the first core component must have minimum and maximum target sizes and tolerances, thereby ensuring proportional serving sizes for each unit or element. The food product may be packaged with single element or packaged with multiplicity of elements. The nutritional claims provided on packaging of the composite food product based on a serving size are thus proportional to an element or unit, and correspond thereto. A specific example follows:

In a preferred embodiment of the present invention, a carrier meat consisting of chicken breast meat is targeted to a unit weight of 83 grams +/−5 grams. The first carrier thus sized is covered or coated with a second nutritionally enhanced breading fortified with niacin, iron, thiamine, riboflavin, folic acid, a calcium salt, and fish oil. Because a consistent amount of breading will adhere to the carrier meat so configured, each element provides 25%, 7.5%, 15%, 7.5%, 10%, and 10% of the US RDA for niacin, iron, thiamine, riboflavin, folic acid, and calcium, respectively, with the maximum difference from the target value of about 6% of the target value for each nutrient.

The amount, shape, and size of the first component varies depending upon the type of meat and type of meal to be provided, such as breakfast, lunch, dinner, or snack.

Thus, the packaging for a nutritionally-enhanced meat product according to the present invention appropriately and properly includes nutritional claims since a serving size is proportional to an element size, which includes a predetermined, controllable amount of the fortified foodstuff component with respect to a predetermined dimension and/or specification of a carrier meat that are separate and distinct from each other.

Either a single element or a multiplicity of elements within a package are both considered as preferable embodiments according to the present invention.

The present invention further includes a method for producing a nutritionally enhanced composite food product comprising the steps of:

providing a meat component having at least one predetermined dimension providing a portion size for providing predictable nutrient levels,

introducing a foodstuff in a predetermined amount, wherein the foodstuff includes at least one nutritional enhancer, each at a predetermined amount, and

combining the foodstuff with the meat component without intermixing.

In a preferred embodiment, the present invention further includes a step of cooking the composite meat product, either fully or partially. The present invention may further include the step of providing a packaging label for the composite meat product that includes nutritional claims directed to the foodstuff and its at least one nutritional enhancer.

Certain modifications and improvements will occur to those skilled in the art upon a reading of the foregoing description. For example, coloration may be included in the fortified foodstuff to enhance the appeal of the food product, e.g., to make a fortified food product that is more appealing to children. All modifications and improvements have been deleted herein for the sake of conciseness and readability but are properly within the scope of the following claims.

We claim:

1. A nutritionally-enhanced composite food product for providing a predictable nutrient amount, comprising at least one serving element, further comprising:
a first carrier meat,
a second nutritionally enhanced foodstuff with at least one nutritional enhancer, the foodstuff stratified from first carrier meat,
the first carrier meat having at least one predetermined dimension providing a portion size for providing predictable nutrient levels of the composite food product,
thereby permitting nutritional claims to be made to the composite food product for inclusion on packaging labeling.

2. The food product as in claim 1 wherein the carrier meat is selected from the group consisting of chicken, turkey, beef, lamb, fish, shellfish, and pork.

3. The food product as in claim 1 wherein the carrier meat is fully cooked.

4. The food product as in claim 1 wherein the nutritionally enhanced foodstuff is exterior to the meat.

5. The food product as in claim 4 wherein the nutritionally enhanced foodstuff is a coating.

6. The food product as in claim 4 wherein the nutritionally enhanced foodstuff is a breading.

7. The food product as in claim 1 wherein the nutritionally enhanced foodstuff is in the interior of the meat.

8. The food product as in claim 7 wherein the nutritionally enhanced foodstuff is surrounded by the carrier meat.

9. The food product as in claim 1 wherein the at least one nutritional enhancer is selected from the group consisting of vitamins, minerals, fatty acids, amino acids, carbohydrates, and proteins.

10. The food product as in claim 1 wherein the at least one nutritional enhancer is a biomediators.

11. The food product as in claim 1 wherein the nutritional enhancers are glucosamine and chondroitin.

12. The food product as in claim 1 wherein the nutritional enhancers are essential fatty acids.

13. The food product as in claim 12 wherein the essential fatty acids are omega-3 fatty acids.

14. The food product as in claim 13 wherein the omega-3 fatty acids are provided by fish oil.

15. The food product as in claim 13 wherein the omega-3 fatty acids are provided by flax seed oil.

16. The food product as in claim 1 wherein a serving size is a multiplicity of elements.

17. The food product as in claim 16 wherein the pre-determined dimension is carrier meat weight.

18. The food product as in claim 17 wherein the elements have predetermined dimensions with tolerances of less than about 6% of the target.

19. The food product as in claim 1 wherein the predetermined dimension is carrier meat surface area.

20. The food product as in claim 19 wherein the elements have predetermined dimensions with tolerances of less than about 6% of the target.

21. The food product as in claim 1 wherein a serving size is a single element.

22. The food product as in claim 1 further including nutritional claims based on a serving size proportional to an element.

23. A nutritionally-enhanced composite meat product for providing a predictable nutrient amount, comprising at least one serving element, further comprising:
a meat component,
a foodstuff with at least one nutritional enhancer, the foodstuff being combined with but distinct from the meat component,
wherein the composite meat product provides at least one predetermined dimension providing a portion size for providing predictable nutrient levels, thereby permitting nutritional claims to be made to the composite food product for inclusion on packaging labeling.

24. The composite meat product according to claim 23, wherein the foodstuff is located on the exterior surface of the meat component.

25. The composite meat product according to claim 23, wherein the meat component surrounds the foodstuff.

26. A method for producing a nutritionally enhanced composite food product comprising the steps of:
providing a meat component having at least one predetermined dimension providing a portion size for providing predictable nutrient levels,
introducing a foodstuff in a predetermined amount, wherein the foodstuff includes at least one nutritional enhancer, each at a predetermined amount, and
combining the foodstuff with the meat component without intermixing.

27. The method according to claim 26, further including the step of cooking the composite meat product.

28. The method according to claim 27, further including the step of providing a packaging label for the composite meat product that includes nutritional claims directed to the foodstuff and its at least one nutritional enhancer.
